(19) United States
(12) Patent Application Publication
(10) Pub. No.: US 2003/0082639 A1
(43) Pub. Date: May 1, 2003

(54) Methods for the identification of herbicides and the modulation of plant growth

(76) Inventors: Keith Davis, Durham, NC (US); Adel Zayed, Durham, NC (US); Robert Ascenzi, Cary, NC (US); Douglas Boyes, Chapel Hill, NC (US); Rao Mulpuri, Apex, NC (US); Neil Hoffman, Chapel Hill, NC (US); Jorn Gorlach, Manchester, NJ (US); Jeffrey Woessner, Hillsborough, NC (US); Carol Hamilton, Apex, NC (US); Kenneth Phillips, Durham, NC (US)

Correspondence Address: PARADIGM GENETICS, INC, 108 ALEXANDER DRIVE, P O BOX 14528, RTP, NC, 27709-4528, US

(21) Appl. No.: 10/282,623

(22) Filed: Oct. 29, 2002

Related U.S. Application Data
(60) Provisional application No. 60/338,462, filed on Oct. 29, 2001.

(51) Int. Cl. G01N 33/53; G01N 33/567; A01N 25/00; A01H 1/00; C12N 15/82

(52) U.S. Cl. 435/7.2; 504/116.1; 800/287

(57) ABSTRACT

The present inventors have discovered that the germin-like polypeptides set forth in SEQ ID NO: 2, 4, 6, 8, and 10 are essential for plant growth. Thus, these polypeptides can be used as targets for the identification of herbicides. Accordingly, the present invention provides methods for the identification of compounds that inhibit the expression or activity of the polypeptides encoded by SEQ ID NO: 2, 4, 6, 8 or 10. Such compounds have use as herbicides. In addition, methods and compositions for modulating plant growth and development are provided.

### RELATED APPLICATIONS

This application claims the priority of U.S. Provisional Patent Application Serial No. 60/338,462, filed on Oct. 29, 2001, the contents of which is incorporated by reference.

### FIELD OF THE INVENTION

The invention relates generally to plant molecular biology. In particular, the invention relates to methods for the identification of herbicides.

### BACKGROUND OF THE INVENTION

The traditional approach to herbicide development may be characterized as “spray and pray.” Chemicals produced in milligram or greater quantity are sprayed on plants and then plant growth is monitored. While this strategy has resulted in the identification of commercially important herbicides, cost, efficacy and safety challenge the future productivity of the “spray and pray” method. Accordingly, there is a need to identify herbicide targets so that compound libraries can be screened for herbicidal activity in high throughput in vitro or cell-based assays. Inhibitors of these targets can then be selected and confirmed as having herbicidal activity in conventional herbicide assays.

Germin is a 130 kDa protein first detected in germinating cereals. Later, this protein was found to be present in cereal cell walls, and still later it was identified as having oxalate oxidase activity. Proteins with sequence identity to germins have been identified from wheat as well as from other plant species, and are called germin-like proteins. Carter et al. (1998) Plant Mol Biol 38:929-43.

The Arabidopsis thaliana genome contains multiple genes that encode germin-like proteins (GLPs). These proteins are so named because they contain several structural elements that are also conserved in wheat germin. The present inventors have discovered that inhibition of the expression of an A. thaliana GLP is detrimental to plant growth. Therefore, GLPs are novel herbicide targets. The present invention provides compositions and methods for the use of GLPs as a novel class of herbicide targets.

### SUMMARY OF THE INVENTION

The present inventors have discovered that antisense expression of a portion of the cDNA of SEQ ID NO: 1, 7 and 9, SEQ ID NO: 7, and SEQ ID NO: 9 in Arabidopsis results in one or more of: absence of leaf formation, delayed development, chlorosis, severe stunting, decreased size, and short roots. Thus, the polypeptides encoded by the cDNA of SEQ ID NO: 1, 7 and 9, SEQ ID NO: 7, and SEQ ID NO: 9 are each essential for normal plant development and growth, and as such each can be used as a target for the identification of herbicides. Accordingly, the present invention provides a method for the identification of herbicide candidates, comprising: contacting a candidate compound with a polypeptide comprising the polypeptide of SEQ ID NO: 2, SEQ ID NO: 8, SEQ ID NO: 1, 7 and 90, or a polypeptide having at least 80% sequence identity with the polypeptides of SEQ ID NO: 2, SEQ ID NO: 8, and SEQ ID NO: 1, 7 and 90 and detecting the presence or absence of binding between said compound and said polypeptide.

In addition, two other proteins SEQ ID NO: 4 (Genbank accession No. CAA63014, from Arabidopsis thaliana) encoded by SEQ ID NO: 3, and SEQ ID NO: 6 (Genbank accession No. AAA86365, from Brassica napus) encoded by SEQ ID NO: 5) are highly homologous to SEQ ID NO: 2, 8 and 10 and are also useful in the methods and compositions of the invention.

In another aspect, the invention provides a method for the identification of herbicide candidates, comprising: contacting a plant cell with a candidate compound and detecting a decrease in the expression of a protein or mRNA selected from the group consisting of: the polypeptide of SEQ ID NO: 2, 4, 6, 8 or 10, a polypeptide having at least 80% sequence identity with the polypeptide of SEQ ID NO: 2, 4, 6, 8 or 10, and an mRNA encoding a polypeptide having at least 80% sequence identity with the polypeptide of SEQ ID NO: 2, 4, 6, 8 or 10. Herbicide candidates identified by these methods can be confirmed as having herbicidal activity using conventional herbicide assays. The methods of the invention are useful for the identification of herbicides.

In another aspect the invention provides a method for identifying a compound as a candidate for a herbicide. The method comprises measuring the auxin-binding activity of a germin-like protein in the absence and presense of the compound. Determining the difference in the auxin-binding activity in the presense and absense of the compound, wherein a difference in the auxin-binding activity indicates the compound as a herbicide candidate. In one embodiment the method comprises contacting a germin-like protein with fluorescein-indole acetic acid in the absence and presense of the compound. Measuring the fluorescence polarization in the presense and absense of the compound, wherein a difference in the fluorescence polarization in the presense and absense of the compound indicates the compound as a herbicide candidate. In one embodiment the germin-like protein is SEQ ID NO: 2, 4, 6, 8 or 10.

In another aspect the invention provides a method for identifying a compound as a candidate for a herbicide. The method comprises measuring the oxalate oxidase activity of a germin-like protein in the absence and presense of the compound. Determining the difference in the oxalate oxidase activity in the presense and absense of the compound, wherein a difference in the oxalate oxidase activity indicates the compound as a herbicide candidate. In one embodiment the method comprises contacting a germin-like protein with oxalate in the absence and presense of the compound. Measuring the H₂O₂ produced in the presense and absense of the compound, wherein a difference in the amount of H₂O₂ in the presense and absense of the compound indicates the compound as a herbicide candidate. In one embodiment the germin-like protein is SEQ ID NO: 2, 4, 6, 8 or 10.

In another aspect the invention provides a method for identifying a compound as a candidate for a herbicide. The method comprises measuring the ADPG pyrophosphatase activity of a germin-like protein in the absence and presense of the compound. Determining the difference in ADPG pyrophosphatase activity in the presense and absense of the compound, wherein a difference in the ADPG pyrophosphatase activity indicates the compound as a herbicide candidate. In one embodiment the germin-like protein is SEQ ID NO: 2, 4, 6, 8 or 10.

In yet another aspect, the invention provides a method for the inhibition of plant growth or the modulation of plant development, comprising expressing antisense RNA complementary to a polynucleotide encoding a polypeptide having at least 80% sequence identity with SEQ ID NO: 2, 4, 6, 8 or 10 in a plant or plant tissue.

In yet another aspect, the invention provides a method for the inhibition of plant growth or the modulation of plant development, comprising expressing a sense RNA polynucleotide encoding a polypeptide having at least 80% sequence identity with SEQ ID NO: 2, 4, 6, 8 or 10 in a plant or plant tissue.

In yet another aspect, the invention provides a method for the inhibition of plant growth or the modulation of plant development, comprising expressing dsRNA specific for a polynucleotide encoding a polypeptide having at least 80% sequence identity with SEQ ID NO: 2, 4, 6, 8 or 10 in a plant or plant tissue.

In yet another aspect, the invention provides a method for the inhibition of plant growth or the modulation of plant development, comprising expressing a ribozyme specific for a polynucleotide encoding a polypeptide having at least 80% sequence identity with SEQ ID NO: 2, 4, 6, 8 or 10 in a plant or plant tissue.

Antisense molecules, sense molecules, dsRNA molecules, ribozymes, expression vectors, transformed plant cells and transgenic plants are also provided.

### DETAILED DESCRIPTION OF THE INVENTION

Definitions

The term “antisense,” for the purposes of the invention, refers to a nucleic acid comprising a polynucleotide which is sufficiently complementary to all or a portion of a gene, primary transcript or processed mRNA, so as to interfere with expression of the endogenous gene.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

“Complementary” polynucleotides are those which are capable of base pairing according to the standard Watson-Crick complementarity rules. Specifically, purines will base pair with pyrimidines to form combinations of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

“Cosuppression” is defined herein as the inhibition of expression of a specific gene in plants by an introduced sense polynucleotide corresponding to the gene.

The term “dsRNA specific for a polynucleotide” is defined as a first ribonucleic acid having at least 80% sequence identity with at least 100 consecutive nucleotides of the polynucleotide encoding either the polypeptide of SEQ ID NO: 2, 4, 6, 8 or 10 or a polypeptide having at least 80% sequence identity with SEQ ID NO: 2, 4, 6, 8 or 10; and a second ribonucleic acid that is substantially complementary to said first ribonucleic acid. Preferably, the first ribonucleic acid of the dsRNA of the invention has at least 80% sequence identity with at least 100 consecutive nucleotides of SEQ ID NO: 1, 3, 5, 7 or 9.

The term “herbicide”, as used herein, refers to a compound that may be used to kill or suppress the growth of at least one plant, plant cell, plant tissue or seed.

By “herbicidally effective amount” is meant an amount of a chemical or composition sufficient to kill a plant or decrease plant growth and/or viability by at least 10%. More preferably, the growth or viability will be decreased by 25%, 50%, 75%, 80%, 90% or more.

For the purposes of the invention, “high stringency hybridization conditions” refers to hybridization in 50% formamide, 1 M NaCl, 1% SDS at 37° C., and a final wash in 0.1×SSC at 60° C. Methods for nucleic acid hybridizations are described in Meinkoth and Wahl (1984) Anal Biochem 138: 267-284 (PMID: 6204550); Current Protocols in Molecular Biology, Chapter 2, Ausubel et al. Eds., Greene Publishing and Wiley—Interscience, New York, 1995; and Tijssen, Laboratory Techniques in Biochemistry and Molecular Biology: Hybridization with Nucleic Acid Probes, Part I, Chapter 2, Elsevier, New York, 1993.

The term “inhibitor,” as used herein, refers to a chemical substance that decreases the expression or the activity of the polypeptide of SEQ ID NO: 2, 4, 6, 8 or 10, or a polypeptide having at least 80% sequence identity with the polypeptide of SEQ ID NO: 2, 4, 6, 8 or 10.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

For the purposes of the invention, an “isolated polynucleotide” is a polynucleotide that is substantially free of the nucleic acid sequences that normally flank the polynucleotide in its naturally occurring replicon. For example, a cloned polynucleotide is considered isolated. Alternatively, a polynucleotide is considered isolated if it has been altered by human intervention, or placed in a locus or location that is not its natural site, or if it is introduced into cell by agroinfection. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide makes up less than 5% of the number of nucleic acid inserts in the vector molecules. Further specifically excluded are whole cell genomic DNA or whole cell RNA preparations (including said whole cell preparations which are mechanically sheared or enzymatically digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

For the purposes of the invention “ligand” is defined as any molecule that exhibits “specific binding” as defined herein.

By “male tissue” is meant the tissues of a plant that are directly involved or supportive of the reproduction of the male gametes. Such tissues include pollen tapetum, anther, tassel, pollen mother cells and microspores. A “male tissue-preferred” or “male tissue-specific” promoter will be expressed predominantly in one or more male tissues. It is possible that a male tissue preferred promoter will be expressed in non-male tissues, however, expression will usually be at a lower level than in male tissues.

“Modulation” is herein defined as an increase, decrease or alteration relative to a control, standard, or reference plant.

As used herein, “nucleic acid” and “polynucleotide” refer to RNA or DNA that is linear or branched, single or double stranded, or a hybrid thereof. The term also encompasses RNA/DNA hybrids. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modifications to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR and in vitro or in vivo transcription.

By “operably linked” is meant that a polynucleotide is functionally linked to a promoter, so that the transcription of the polynucleotide can be initiated from the promoter.

For the purposes of the invention, the “percent (%) sequence identity” between two polynucleotide or two polypeptide sequences is determined according to the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings). It is 10 understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

As used herein, the term “probe” is a polynucleotide having a defined sequence with no more than 10 additional nucleic acid residues at either of its ends.

For the purposes of the invention, “recombinant polynucleotide” refers to a polynucleotide that has been altered, rearranged or modified by genetic engineering. Examples include any cloned polynucleotide, and polynucleotides that are linked or joined to heterologous sequences. Two polynucleotide sequences are heterologous if they are not naturally found joined together. The term recombinant does not refer to alterations to polynucleotides that result from naturally occurring events, such as spontaneous mutations.

By “ribozyme” is meant a catalytic RNA-based enzyme capable of targeting and cleaving particular base sequences in both DNA and RNA. Ribozymes comprise a polynucleotide sequence that is complementary to a portion of a target nucleic acid and a catalytic region that cleaves the target nucleic acid. Ribozymes can be designed that specifically pair with and inactivate a target RNA by catalytically cleaving the RNA at a targeted phosphodiester bond. Methods for making and using ribozymes are known to those skilled in the art. See, for example, U.S. Pat. Nos. 6,025,167; 5,773,260 and 5,496,698, the contents of which are incorporated by reference, and Haseloff and Gerlach (1988) Nature 334: 586-591 (PMID: 2457170).

For the purposes of the invention “a ribozyme that is specific for a polynucleotide” is defined as a ribozyme capable of targeting and cleaving at least one phosphodiester bond in the polynucleotide selected from the group consisting of: the polynucleotide of SEQ ID NO: 1, 3, 5, 7 or 9, a polynucleotide having at least 80% sequence identity with SEQ ID NO: 1, 3, 5, 7 or 9 , a polynucleotide encoding the polypeptide of SEQ ID NO: 2, 4, 6, 8 or 10, and a polynucleotide encoding a polypeptide having at least 80% sequence identity to SEQ ID NO: 2, 4, 6, 8 or 10. Preferably, the ribozyme is specific for the polynucleotide encoded by SEQ ID NO: 1, 7 and 9.

The term “specific binding” refers to an interaction between the polypeptide of SEQ ID NO: 2, 4, 6, 8 or 10, a polypeptide having at least 80% sequence identity with the polypeptide of SEQ ID NO: 2, 4, 6, 8 or 10, or a polypeptide comprising at least 10 consecutive amino acid residues of the polypeptide of SEQ ID NO: 2, 4, 6, 8 or 10, and a molecule or compound, wherein the interaction is dependent upon the primary amino acid sequence or the conformation of said polypeptide.

By “substantially complementary,” is meant that when two hybridizing RNAs are optimally aligned using the BLAST program as described herein, the hybridizing portions are at least 95% complementary.

“Transform,” as used herein, refers to the introduction of a polynucleotide (single or double stranded DNA, RNA, or a combination thereof) into a living cell by any means. Transformation may be accomplished by a variety of methods, including, but not limited to, agroinfection, electroporation, particle bombardment, and the like. This process may result in transient or stable (constitutive or regulated) expression of the transformed polynucleotide. By “stably transformed” is meant that the sequence of interest is integrated into a replicon in the cell, such as a chromosome or episome. Transformed cells, tissues and plants encompass not only the end product of a transformation process, but also the progeny thereof which retain the polynucleotide of interest.

For the purposes of the invention, “transgenic” refers to any plant, plant cell, callus, plant tissue or plant part, that contains all or part of at least one recombinant polynucleotide. In many cases, all or part of the recombinant polynucleotide is stably integrated into a chromosome or stable extra-chromosomal element, so that it is passed on to successive generations.

The present inventors have discovered that antisense expression of RNA complementary to a portion of the cDNA of SEQ ID NOS: 1, 7, and 9 strongly inhibits the growth and development of Arabidopsis seedlings. The cDNA of SEQ ID NO: 1, 7 and 9 encode the polypeptides of SEQ ID NO: 2, 8, and 10, respectively. SEQ ID NOS: 1, 2, 7, 8, 9, and 10 have been reported in the prior art (see TIGR database locus At1g72610, At5g39110, and At5g61750). However, heretofore, SEQ ID NO: 1, 7 and 9 had not been identified as herbicide targets. Thus, the inventors are the first to demonstrate that the polynucleotides of SEQ ID NO: 1, 7 and 9 and the polypeptides of SEQ ID NO: 2, 8 and are targets for herbicides. In addition, the homologous polypeptides of SEQ ID NO: 4 and SEQ ID NO: 6 are also useful to identify herbicide targets.

In one aspect, the invention provides methods for identifying compounds that inhibit the expression or activity of the polypeptide of SEQ ID NO: 2, 4, 6, 8 or 10. Such methods include ligand binding assays, enzyme activity assays and assays for RNA or protein expression. Any compound that is a ligand for the polypeptide of SEQ ID NO: 2, 4, 6, 8 or 10 may have herbicidal activity. Polypeptides having at least 80% sequence identity with the polypeptide of SEQ ID NO: 2, 4, 6, 8 or 10 can also used in the methods of the invention to identify herbicide candidates. Preferably, the sequence identity with SEQ ID NO: 2, 4, 6, 8 or 10 is at least 85%, 90% or 93%, more preferably the identity is at least 95%, most preferably the sequence identity is at least 96%, 97%, 98% or 99%.

Thus, in one embodiment, the invention provides a method for identifying a compound as a herbicide, comprising: selecting a compound that binds to the polypeptide selected from the group consisting of: the polypeptide of SEQ ID NO: 2, 4, 6, 8 or 10 and a polypeptide having at least 80% sequence identity with the polypeptide of SEQ ID NO: 2, 4, 6, 8 or 10; and contacting a plant with said compound to confirm herbicidal activity.

In another embodiment, the invention provides a method for identifying herbicide candidates, comprising: contacting a compound with a polypeptide selected from the group consisting of:

i) the polypeptide of SEQ ID NO: 2, 4, 6, 8 or 10; and

ii) a polypeptide having at least 80% sequence identity with the polypeptide of SEQ ID NO: 2, 4, 6, 8 or 10; and detecting the presence and/or absence of binding between said compound and said polypeptide; wherein binding indicates that said compound is a herbicide candidate.

The polypeptide of SEQ ID NO: 2, 8, or 10 is contacted with a test compound in the ligand-binding assay described above. The polypeptide of SEQ ID NO: 2, 8, or 10 is encoded by the cDNA of SEQ ID NO: 1, 7 and 9, respectively. One skilled in the art could determine any or all of the additional polynucleotides that could encode the polypeptide of SEQ ID NO: 2, 8, and 10. In addition, the polynucleotide of SEQ ID NO: 1, 7 and 9 can be used as a probe to isolate cDNAs or genes that encode a polypeptide having at least 80% sequence identity with the polypeptide of SEQ ID NO: 2, 4, 6, 8 or 10.

Polypeptides having at least 80% sequence identity to the polypeptide of SEQ ID NO: 2, 4, 6, 8 or 10 can correspond to naturally occuring polypeptides from any organism, or can be synthetic or recombinant variants of naturally occuring polypeptides. Preferably, the polypeptide is from a plant or a microorganism, such as bacteria or fungi. Most preferably the polypeptide is from a plant.

In one embodiment, the polypeptide is from Arabidopsis. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii.

In other embodiments, the polypeptide is from a weed. For example, the polypeptide having at least 80% sequence identity with the polypeptide of SEQ ID NO: 2, 4, 6, 8 or 10 can be from weeds including, but not limited to, barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of the polypeptide of SEQ ID NO: 4, 6, 8 or 10 may be used in the methods of the invention. The fragments comprise at least 10 consecutive amino acids of the polypeptide of SEQ ID NO: 4, 6, 8 or 10. Preferably, the fragment comprises at least 15, 20, 25, 30, 35, 40, 50, 60, 70, 80, 90 or at least 100 consecutive amino acids residues of SEQ ID NO: 4, 6, 8 or 10.

For the ligand binding assays, the polypeptide of SEQ ID NO: 4, 6, 8 or 10 and polypeptides having at least 80% sequence identity with the polypeptide of SEQ ID NO: 4, 6, 8 or 10, and fragments thereof may be purified from a plant or may be recombinantly produced in and purified from a plant, bacteria, or eukaryotic cell culture. Preferably these proteins are produced using a baculovirus or E. coli expression system. Methods for protein expression and purification using these and other systems are well known to those skilled in the art.

Any compound may be screened for herbicidal activity using the methods of the invention. Examples of compounds that could be screened include inorganic and organic compounds such as, but not limited to, amino acids, peptides, proteins, nucleotides, nucleic acids, glyco-conjugates, oligosaccharides, lipids, alcohols, thiols, aldehydes, alkylators, carbonic ethers, hydrazides, hydrazines, ketons, nitrils, amines, sulfochlorides, triazines, piperizines, sulphonamides and the like. Preferably compound libraries are screened in the assays of the invention. Methods for synthesizing and screening compound libraries are known to those skilled in the art. See for example, U.S. Pat. Nos. 5,463,564; 5,574,656; 5,684,711; and 5,901,069, the contents of which are incorporated by reference.

Any technique for detecting the binding of a ligand to its target may be used in the methods of the invention. Polypeptides and proteins that can reduce non-specific binding, such as BSA, or protein extracts from cells that do not produce the target, may be included in the binding assay. Many methods for detecting the binding of a ligand to its target are known in the art, and include, but are not limited to the detection of an immobilized ligand-target complex or the detection of a change in a physical property of a target when it is bound to a ligand.

In one embodiment, an array of immobilized candidate ligands is provided. The immobilized ligands are contacted with the polypeptide of SEQ ID NO: 4, 6, 8 or 10, a polypeptide having at least 80% sequence identity with the polypeptide of SEQ ID NO 4, 6, 8 or 10, or a fragment or variant thereof, the unbound protein is then removed and the bound polypeptide is detected. In a preferred embodiment, bound polypeptide is detected using a labeled binding partner, such as a labeled antibody. Methods for making antibodies to polypeptides are well known to those skilled in the art. Preferred labels include fluorescent or radioactive moieties. In another embodiment, the polypeptide of SEQ ID NO: 4, 6, 8 or 10, or a fragment or variant thereof, is labeled prior to contacting the immobilized candidate ligands. Preferred detection methods include fluorescence correlation spectroscopy (FCS) and FCS-related confocal nanofluorimetric methods. See Rigler (1995) J Biotechnol 41:177-86. In another embodiment, the assay may be performed as described in Zhang et al. (1996) Plant Molecular Biology Reporter 14:266-72.

In another embodiment, the immobilized polypeptide of SEQ ID NO: 4, 6, 8 or 10, or a polypeptide having at least 80% sequence identity with the polypeptide of SEQ ID NO: 4, 6, 8 or 10, or a fragment or variant thereof, is contacted with a candidate compound library. Specific binding to the target polypeptide can be detected by various methods known in the art including affinity selection chromatography, ultrafiltration assays, the scintillation proximity assay, interfacial optical techniques (surface plasmon resonance and its relatives), and the like. See Woodbury and Venton (1999) J Chromatogr B Biomed Sci Appl 2:113-137.

In another method, in which the target polypeptide is not adsorbed to a matrix, target-ligand binding is detected using mass spectroscopy, such as Matrix-Assisted Laser Desorption Ionization Time-Of-Flight (MALDI-TOF) analysis. Bonk and Humeny (2001) Neuroscientist 7:6-12. MALDI-TOF is capable of detecting and identifying the binding of ligands such as, but not limited to, peptides, proteins, nucleic acids, glyco-conjugates, oligosaccharides, organic polymers and the like.

Once a compound is identified as a candidate for a herbicide or has been selected as binding to the polyeptide of SEQ ID NO: 4, 6, 8 or 10, or variants thereof, it can be tested for herbicidal activity by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by a method of the invention has herbicidal activity, comprising: contacting a plant or plant cells with said herbicide candidate and detecting the presence or absence of a decrease in the growth or viability of said plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

As an alternative to in vitro assays, the invention also provides plant and plant cell based assays for detecting target RNA or protein expression in the presence and absence of a test compound. The target RNA may be a primary RNA transcript or a processed mRNA. In a preferred embodiment, the mRNA corresponds to the cDNA of SEQ ID NO: 1, 3, 5, 7 or 9. For the purposes of the invention, an RNA sequence corresponds to a DNA sequence when the sequences are the same, except that the thymine nucleotides of the DNA are replaced by uracil nucleotides in the RNA. In one embodiment, the mRNA has at least 80%, 85%, 90%, 93%, 95%, 96%, 97%, 98% or even 99% sequence identity with SEQ ID NO: 1, 3, 5, 7 or 9. In an alternative embodiment, the mRNA measured encodes the polypeptide of SEQ ID NO: 4, 6, 8 or 10 or a polypeptide having at least 80%, 85%, 90%, 93%, 95%, 96%, 97%, 98% or even 99% sequence identity with the polypeptide of SEQ ID NO: 4, 6, 8 or 10.

Thus, the invention provides a method for identifying a compound as a candidate for a herbicide, comprising:

a) measuring the expression of an RNA in a plant or plant cell in the presence and absence of said compound, wherein said RNA is selected from the group consisting of:

i) an MRNA corresponding to the cDNA of SEQ ID NO: 1, 3, 5, 7 or 9;

ii) an RNA having at least 80% sequence identity with the cDNA of SEQ ID NO: 1, 3, 5, 7 or 9;

iii) an RNA encoding the polypeptide of SEQ ID NO: 2, 4, 6, 8 or 10; and

iv) an RNA encoding a polypeptide having at least 80% sequence identity to the polypeptide of SEQ ID NO: 2, 4, 6, 8 or 10; and

b) comparing the expression of said RNA in the presence and absence of said compound, wherein a decrease in the expression of said RNA in the presence of said compound indicates that said compound is a herbicide candidate.

Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Such methods include, but are not limited to amplification assays such as quantitative PCR, and/or hybridization assays such as Northern analysis, dot blots, slot blots, in-situ hybridization, bDNA assays and microarray assays.

In another embodiment, the invention provides a method for identifying a compound as a candidate for a herbicide, comprising:

a) measuring the expression of a protein in a plant or plant cell in the presence and absence of said compound, wherein said protein is selected from the group consisting of:

i) the polypeptide of SEQ ID NO: 2, 4, 6, 8 or 10; and

ii) a polypeptide having at least 80% sequence identity with the polypeptide of SEQ ID NO: 2, 4, 6, 8 or 10; and

b) comparing the expression of said protein in the presence and absence of said compound, wherein a decrease in the expression of said protein in the presence of said compound indicates that said compound is a herbicide candidate.

Preferably the polypeptide is the polypeptide of SEQ ID NO: 2, 4, 6, 8 or 10. Alternatively, the polypeptide has at least 80%, 85%, 90%, 93%, 95%, 96%, 97%, 98% or even 99% sequence identity with the polypeptide of SEQ ID NO: 2, 4, 6, 8 or 10.

Methods for detecting protein expression include, but are not limited to, immunodetection methods such as Western blots, ELISA assays, polyacrylamide gel electrophoresis, mass spectroscopy and enzymatic assays. In one embodiment, an in situ assay such as those described in Zhou et al. (1998) Plant Physiology 117:33-41 and Dumas et al. (1995) Plant Physiology 107:1091-1096 may be used.

Also, any reporter gene system may be used to detect protein expression. For detection using gene reporter systems, a polynucleotide encoding a reporter protein is fused in frame with a polynucleotide encoding the polypeptide of SEQ ID NO: 2, 4, 6, 8 or 10, or a variant or fragment thereof, so as to produce a chimeric polypeptide. Preferably, expression of the chimeric polypeptide is under the control of the cognate promoter that regulates expression of an mRNA corresponding to SEQ ID NO: 1, 3, 5, 7 or 9. This promoter could be obtained by using SEQ ID NO: 1, 3, 5, 7 or 9 as a probe to identify a clone in a genomic library containing at least the 5′ portion of the gene encoding SEQ ID NO: 2, 4, 6, 8 or 10. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107-111), β-galactosidase (Nolan et al (1988) Proc Natl Acad Sci USA 85:2603-2607), alkaline phosphatase (Berger et al. (1988) Gene 66:10), luciferase (De Wet et al. (1987) Mol Cell Biol 7:725-737), β-glucuronidase (GUS), fluorescent proteins, chromogenic proteins and the like.

The herbicidal activity of compounds identified as herbicide candidates by the RNA and protein expression methods described above can be confirmed by contacting a plant or plant cells with the herbicide candidate and detecting the presence or absence of a decrease in growth or viability of said plant or plant cells.

Compounds identified as herbicides can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Having identified the expression and activity of the polypeptide of SEQ ID NO: 2, 8 and 10 as essential for plant growth and development, the invention provides compounds for the inhibition and modulation of plant growth. As described herein, antisense expression of a portion of an RNA complementary to the cDNA of SEQ ID NO: 1, 7 and 9 in plant seedlings results in extremely poor growth and developmental abnormalities. Accordingly, the invention provides polynucleotides that specifically inhibit the expression of the polypeptide of SEQ ID NO: 2, 8 and 10 and related polypeptides such as SEQ ID NO: 4 and SEQ ID NO: 6.

The polynucleotides of the invention are capable of specifically inhibiting transcription or translation, or decreasing the stability of a polynucleotide encoding the polypeptide of SEQ ID NO: 2, 4, 6, 8 or 10 and polypeptides having at least 80% sequence identity with SEQ ID NO: 2, 4, 6, 8 or 10. Such polynucleotides include, but are not limited to, antisense molecules, ribozymes, sense molecules, interfering double-stranded RNA (dsRNA) and the like.

The effect of the expression of such polynucleotides on plant growth and development will depend upon many factors, such as the specificity and activity of the polynucleotide, the level of expression of the polynucleotide and the expression pattern of the promoter driving the expression of a polynucleotide of the invention. For example, inducible expression of such polynucleotides can result in plant death, decreased plant size or decreased growth at the time of induction. Similarly, developmentally regulated expression could result in a reduction of growth or plant death at a particular stage of development.

Tissue specific expression will result in necrosis or reduced growth of that tissue. In preferred embodiments, the polynucleotides of the invention are operably linked to a tissue-specific or tissue preferred promoter. In one embodiment, the polynucleotides of the invention are operably linked to a male-tissue preferred promoter. Male tissue-preferred expression of a polynucleotide of the invention can result in male-sterile plants. Female tissue-preferred expression of a polynucleotide of the invention can result in seedless plants, or in plants having reduced seed size.

While the polynucleotides of the invention are not limited to a particular mechanism of action, reduction in gene expression can be mediated at the DNA level and at transcriptional, post-transcriptional, or translational levels. For example, it is thought that dsRNA suppresses gene expression by both a posttranscriptional process and by DNA methylation. Sharp and Zamore (2000) Science 287: 2431-33 (PMID: 10766620). Ribozymes specifically bind and catalytically cleave RNA. Gene specific inhibition of expression in plants by an introduced sense polynucleotide is termed “cosuppression”. Antisense polynucleotides, when introduced into a plant cell, are thought to specifically bind to their target polynucleotide and inhibit gene expression by interfering with transcription, splicing, transport, translation and/or stability. Reported mechanisms of antisense action include RNase H-mediated cleavage, activation or inhibition of splicing, inhibition of 5′-cap formation, translation arrest and activation of double strand RNases. See Crooke (1999) Biochim Biophys Acta 1489: 31-44 (PMID: 10806995). Antisense polynucleotides can be targeted to chromosomal DNA, to a primary RNA transcript or to a processed mRNA. Preferred target regions include splice sites and translation initiation and termination codons, and other sequences within the open reading frame.

Thus, the invention provides an isolated antisense RNA for modulating plant growth, comprising, an RNA selected from the group consisting of:

a) an RNA complementary to SEQ ID NO: 1, 7 and 9;

b) an RNA complementary to at least 20 consecutive nucleotides of SEQ ID NO: 1, 7 and 9;

c) an RNA complementary to a polynucleotide having at least 80% sequence identity with SEQ ID NO: 1, 7 and 9, 3, or 5;

d) an RNA complementary to at least 30 consecutive nucleotides of a polynucleotide encoding SEQ ID NO: 2, 4, 6, 8 or 10; and

e) an RNA complementary to a polynucleotide encoding a polypeptide having at least 80% sequence identity with SEQ ID NO: 2, 4, 6, 8 or 10.

In preferred embodiments, the polynucleotide is complementary to a plant mRNA. Preferably, the antisense RNA is complementary to at least 20, 30, 40, 50, 75, 100, 150 or 200 consecutive nucleotides of SEQ ID NO: 1, 3, 5, 7 or 9 or other polynucleotide encoding SEQ ID NO: 2, 4, 6, 8 or 10. In another embodiment, the antisense RNA is complementary to a polynucleotide having at least 80%, 85%, 90%, 93%, 95%, 97%, 98% or even 99% sequence identity with SEQ ID NO: 1, 3, 5, 7 or 9 or other polynucleotide encoding SEQ ID NO: 2, 4, 6, 8 or 10.

In another aspect, the invention provides antisense molecules that specifically hybridize under high stringency conditions to SEQ ID NO: 1, 3, 5, 7 or 9 or a polynucleotide encoding SEQ ID NO: 2, 4, 6, 8 or 10. By “specifically hybridize” is meant that the polynucleotide will hybridize to the target gene or RNA at a level of at least two-fold over background under conditions of high stringency. The specificity of the hybridization will depend upon many factors, including the length and degree of complementarity between the antisense molecule and the target sequence, the length of the antisense molecule, the temperature of the hybridizations and washes, and the salt, detergent and formamide concentrations of the hybridization and wash buffers.

It is understood that the antisense polynucleotides of the invention need not be completely complementary to the target gene or RNA, nor that they hybridize to each other along their entire length, in order to modulate expression or to form specific hybrids. Furthermore, the antisense polynucleotides of the invention need not be full length with respect to the target gene or RNA. In general, greater homology can compensate for shorter polynucleotide length.

Typically such antisense molecules will comprise an RNA having 60-100 % sequence identity with at least 14, 15, 16, 17, 18, 19, 20, 25, 30, 50, 75 or at least 100 consecutive nucleotides of to SEQ ID NO: 1, 3, 5, 7 or 9or a polynucleotide encoding SEQ ID NO: 2, 4, 6, 8 or 10. Preferably, the sequence identity will be at least 70%, more preferably at least 75%, 80%, 85%, 90%, 95%, 98% and most preferably at least 99%.

The active antisense molecules of the invention are single stranded RNA molecules. By active antisense molecule is meant that the antisense RNA is capable of selectively hybridizing with a primary transcript or mRNA encoding a polypeptide having at least 80% sequence identity with the polypeptide of SEQ ID NO: 2, 4, 6, 8 or 10. However, it is understood that the term antisense molecules include double-stranded DNA expression cassettes that can be transcribed to produce an antisense RNA.

Preferably, the antisense polynucleotides of the invention are at least 8, 10, 12, 14, 16, 18, 20, 25, 30, 35, 40, 45, 50, 100, 200, 500, 600 nucleotides or more. Antisense polynucleotides can be selected based on complementarity to plant genes or RNAs. The complementarity may be to all or a portion of the gene or RNA. Furthermore, the complementarity need not be exact, so long as the antisense molecule is specific for the target RNA. In general, the degree of complementarity necessary or antisense inhibition is related to the length of the hybridizing sequences. Preferably, the complementarity is at least 90%, more preferably 95%, even more preferably at least 98% and most preferably 100%. Antisense polynucleotides may be designed to bind to exons, introns, exon-intron boundaries, the promoter and other control regions, such as the transcription and translational initiation sites. Methods for inhibiting plant gene expression using antisense RNA corresponding to entire and partial cDNA, 3′ non-coding regions, as well as relatively short fragments of coding regions are known in the art. See, for example, U.S. Pat. Nos. 5,107,065 and 5,254,800, the contents of which are incorporated by reference, Sheehy et al. (1988) Proc Natl Acad Sci USA 85: 8805-9; Cannon et al. (1990) Plant Mol Biol 15: 39-47 (PMID: 2103441); and Ch'ng et al. (1989) Proc Natl Acad Sci USA 86:10006-10 (PMID: 2481308). Van der Krol et al. (1988) Biotechniques 6: 958-76 (PMID: 2483657) describe the use of antisense RNA to inhibit plant genes in a tissue-specific manner.

As an alternative to antisense polynucleotides, ribozymes, sense polynucleotides or dsRNA may be used to reduce expression of a polypeptide having at least 80% sequence identity with SEQ ID NO: 2, 4, 6, 8 or 10. A ribozyme, or catalytic RNA can catalyze the hydrolysis of RNA phosphodiester bonds in trans, and thus can cleave other RNA molecules. Cleavage of a target RNA can decrease stability of the RNA and prevent translation of a full length protein encoded by that RNA.

Ribozymes contain a first RNA sequence that is complementary to a target RNA linked to a second enzymatic RNA sequence that catalytically cleaves the target RNA. Thus, the ribozyme first binds a target RNA through complementary base-pairing, and then acts enzymatically to cut the target RNA. Ribozymes may be designed to bind to exons, introns, exon-intron boundaries and control regions, such as the translational initiation sites.

At least six types of naturally-occurring enzymatic RNAs, including hairpin ribozymes and hammerhead ribozymes, have been described. The hairpin ribozyme can be assembled in various combinations to catalyze a unimolecular, bimolecular or a trimolecular cleavage/ligation reaction (Berzal-Herranz et al. (1992) Genes & Develop 6: 129 (PMID: 1730406); Chowrira and Burke (1992) Nucleic Acids Res 20:2835 (PMID: 1377380); Komatsu et al (1993) Nucleic Acids Res 21:185 (PMID: 8441626); Komatsu et al. (1994) J Am Chem Soc 116: 3692). Increasing the length of helix 1 and helix 4 regions do not affect the catalytic activity of the hairpin ribozyme (Hisamatsu et al., supra; Chowrira and Burke, supra; Anderson et al. (1994) Nucleic Acids Res 22: 1096 (PMID: 8152912)). For a review of various ribozyme motifs, and hairpin ribozyme in particular, see Ahsen and Schroeder (1993) Bioessays 15: 299; Cech (1992) Curr Opi Struc Bio 2: 605; and Hampel et al. (1993) Methods: A Companion to Methods in Enzymology 5: 37.

The invention provides ribozymes that are specific for at least one RNA encoding a polypeptide having at least 80% sequence identity with SEQ ID NO: 2, 4, 6, 8 or 10. A ribozyme that is “specific for at least one plant RNA encoding a polypeptide having at least 80% sequence identity with SEQ ID NO: 2, 4, 6, 8 or 10” will contain a polynucleotide sequence that specifically hybridizes to a target plant primary transcript or mRNA (the “target”) encoding a polypeptide having at least 80% sequence identity with SEQ ID NO: 2, 4, 6, 8 or 10 and cleaves that target. The portion of the ribozyme that hybridizes to the transcript or RNA is typically at least 7 nucleotides in length. Preferably, this portion is at least 8, 9, 10, 12, 14, 16, 18 or 20 or more nucleotides in length. The portion of the ribozyme that hybridizes to the target need not be completely complementary to the target, as long as the hybridization is specific for the target. In preferred embodiments the ribozyme will contain a portion having at least 7 or 8 nucleotides that have 100% complementarity to a portion of the target RNA. In one embodiment, the target RNA corresponds to the cDNA of SEQ ID NO: 1, 7 and 9.

Methods for designing and preparing ribozymes are known to those skilled in the art. See, for example, U.S. Pat. Nos. 6,025,167; 5,773,260; 5,695,992; 5,545,729; 5,496,698 and 4,987,071, the contents of which are incorporated by reference; Van Tol et al. (1991) Virology 180: 23 (PMID: 1984650); Hisamatsu et al. (1993) Nucleic Acids Symp Ser 29: 173 (PMID: 7504243); Berzal-Herranz et al. (1993) EMBO J 12: 2567 (PMID: 8508779) (describing essential nucleotides in the hairpin ribozyme); Hampel and Tritz, (1989) Biochemistry 28: 4929 (PMID: 2765519); Haseloffet al. (1988) Nature 334: 585-91 (PMID: 2457170), Haseloff and Gerlach (1989) Gene 82: 43 (PMID: 2684775) (describing sequences required for self-cleavage reactions); and Feldstein et al. (1989) Gene 82: 53 (PMID: 2583519).

In another aspect, the invention provides a double-stranded RNA (dsRNA) that is specific for a polynucleotide encoding either the polypeptide of SEQ ID NO: 2, 4, 6, 8 or 10 or a polypeptide having at least 80% sequence identity with SEQ ID NO: 2, 4, 6, 8 or 10. The term dsRNA, as used herein, refers to RNA hybrids comprising two strands of RNA. The dsRNAs of the invention may be linear or circular in structure. The hybridizing RNAs may be substantially or completely complementary. By substantially complementary, is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs of will be of identical length with no overhanging 5′ or 3′ ends and no gaps. However, dsRNAs having 5′ or 3′ overhangs of up to 100 nucleotides may be used in the methods of the invention.

Thus, in one embodiment, the invention provides a dsRNA, comprising: a first ribonucleic acid having at least 80% sequence identity with at least 100 consecutive nucleotides of a polynucleotide encoding either the polypeptide of SEQ ID NO: 2, 4, 6, 8 or 10 or a polypeptide having at least 80% sequence identity with SEQ ID NO: 2, 4, 6, 8 or 10; and a second ribonucleic acid that is substantially complementary to said first ribonucleic acid. Such a dsRNA is specific for a polynucleotide encoding a polypeptide having at least 80% sequence identity with SEQ ID NO: 2, 4, 6, 8 or 10.

Preferably, the first ribonucleic acid of the dsRNA of the invention has at least 80% sequence identity with at least 100 consecutive nucleotides of SEQ ID NO: 1, 3, 5, 7 or 9. Alternatively, the second ribonucleic acid hybridizes to SEQ ID NO: 1, 3, 5, 7 or 9 under high stringency conditions.

The dsRNA may comprise ribonucleotides or ribonucleotide analogs, such as 2′-O-methyl ribosyl residues or combinations thereof. See U.S. Pat. Nos. 4,130,641 and 4,024,222. A dsRNA polyriboinosinic acid:polyribocytidylic acid is described in U.S. Pat. No. 4,283,393.

Methods for making and using dsRNA are known in the art. One method comprises the simultaneous transcription of two complementary DNA strands, either in vivo, or in a single in vitro reaction mixture. See, for example, U.S. Pat. No. 5,795,715, the content of which is incorporated by reference. dsRNA can be introduced into a plant or plant cell directly by standard transformation procedures. Alternatively, dsRNA can be expressed in a plant cell by transcribing two complementary RNAs.

Other methods for the inhibition of endogenous gene expression, such as triple helix formation (Moser and Dervan (1987) Science 238: 645-50 (PMID: 3118463) and Cooney et al. (1988) Science 241: 456-9 (PMID: 3293213)) and cosuppression (Napoli et al. (1990) The Plant Cell 2: 279-89) are known in the art. Partial and full-length cDNAs have been used for the cosuppression of endogenous plant genes. See, for example, U.S. Pat. Nos. 4,801,340, 5,034,323, 5,231,020 and 5,283,184, the contents of which are incorporated by reference, Van der Kroll et al. (1990) The Plant Cell 2: 291-9, Smith et al (1990) Mol Gen Genetics 224: 477-81 and Napoli et al. (1990) The Plant Cell 2: 279-89.

For sense suppression, it is believed that introduction of a sense polynucleotide blocks transcription of the corresponding target gene. The sense polynucleotide will have at least 65% sequence identity with the target plant gene or RNA. Preferably, the percent identity is at least 80%, 90%, 95% or more. The introduced sense polynucleotide need not be full length relative to the target gene or transcript. Preferably, the sense polynucleotide will have at least 65% sequence identity with at least 100 consecutive nucleotides of SEQ ID NO: 1, 3, 5, 7 or 9. The regions of identity can comprise introns and and/or exons and untranslated regions. The introduced sense polynucleotide may be present in the plant cell transiently, or may be stably integrated into a plant chromosome or extrachromosomal replicon.

Expression of the polynucleotides of the invention in a plant, plant cell or plant tissue will result in the modulation of plant growth and/or development. Accordingly, the invention provides recombinant expression cassettes, comprising the antisense, sense, dsRNA or ribozyme polynucleotides of the invention, wherein said polynucleotide is operably linked to a promoter that can be active in a plant cell.

The expression cassettes of the invention contain 5′ and 3′ regulatory sequences necessary for transcription and termination of the polynucleotide of interest. Thus, the expression cassettes will include a promoter and a transcriptional terminator. Other functional sequences may be included in the expression cassettes of the inventions. Such functional sequences include, but are not limited to, introns, enhancers and translational initiation and termination sites and polyadenylation sites. The control sequences can be those that can function in at least one plant, plant cell or plant tissue. These sequences may be derived form one or more genes, or can be created using recombinant technology.

Promoters useful in the expression cassettes of the invention include any promoter that is capable of initiating transcription in a plant cell. Such promoters include, but are not limited to those that can be obtained from plants, plant viruses and bacteria that contain genes that are expressed in plants, such as Agrobacterium and Rhizobium.

The promoter may be constitutive, inducible, developmental stage-preferred, cell type-preferred, tissue-preferred or organ-preferred. Constitutive promoters are active under most conditions. Examples of constitutive promoters include the CaMV 19S and 35 S promoters (Odell et al. (1985) Nature 313: 810-12 (PMID: 3974711)), the 2×CaMV 35S promoter (Kay et al. (1987) Science 236: 1299-1302) the Sep1 promoter, the rice actin promoter (McElroy et al. (1990) Plant Cell 2: 163-71 (PMID: 2136633)), the Arabidopsis actin promoter, the ubiquitan promoter (Christensen et al. (1989) Plant Molec Biol 18: 675-89); pEmu (Last et al. (1991) Theor Appl Genet 81: 581-8), the figwort mosaic virus 35S promoter, the Smas promoter (Velten et al (1984) EMBO J 3: 2723-30), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

Inducible promoters are active under certain environmental conditions, such as the presence or absence of a nutrient or metabolite, heat or cold, light, pathogen attack, anaerobic conditions, and the like. For example, the hsp80 promoter from Brassica is induced by heat shock, the PPDK promoter is induced by light, the PR-1 promoter from tobacco, Arabidopsis and maize are inducible by infection with a pathogen, and the Adh1 promoter is induced by hypoxia and cold stress.

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like.

In a preferred embodiment, the promoter is a male tissue-preferred promoter. Male tissues include pollen, tapetum, anther, tassel, pollen mother cells and microspores. Ms45 is an example of a male-preferred promoter (U.S. Pat. No. 6,037,523). Other tissue preferred, developmental stage preferred and/or inducible promoters include, but are not limited to Prha (expressed in root, seedling, lateral root, shoot apex, cotyledon, petiol, inflorescence stem, flower, stigma, anthers, and silique, and auxin-inducible in roots); VSP2 (expressed in flower buds, flowers, and leaves, and wound inducible); SUC2 (expressed in vascular tissue of cotyledons, leaves and hypocotyl phloem, flower buds, sepals and ovaries); AAP2 (silique-preferred); SUC1 (Anther and pistil preferred); AAP1 (seed preferred); Saur-AC1 (auxin inducible in cotyledons, hypocotyl and flower); Enod 40 (expressed in root, stipule, cotyledon, hypocotyl and flower); amd VSP1 (expressed in young siliques, flowers and leaves).

Seed preferred promoters are preferentially expressed during seed development and/or germination. For example, seed preferred promoters can be embryo-preferred, endosperm preferred and seed coat-preferred. See Thompson and Larkins (1989) BioEssays 10: 108 (PMID: 2658986). Examples of seed preferred promoters include, but are not limited to cellulose synthase (celA), Cim1, gamma-zein, globulin-1, maize 19 kD zein (cZ19B1) and the like.

Other promoters useful in the expression cassettes of the invention include, but are not limited to, the major chlorophyll a/b binding protein promoter, histone promoters, the prolifera promoter, the Ap3 promoter, the β-conglycin promoter, the phaseolin promoter, the napin promoter, the soy bean lectin promoter, the maize 15 kD zein promoter, the 22 kD zein promoter, the 27 kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546) and the SGB6 promoter (U.S. Pat. No. 5,470,359), as well as synthetic or other natural promoters.

Additional flexibility in controlling heterologous gene expression in plants may be obtained by using DNA binding domains and response elements from heterologous sources (i.e., DNA binding domains from non-plant sources). Some examples of such heterologous DNA binding domains include the LexA and GAL4 DNA binding domains. The LexA DNA-binding domain is part of the repressor protein LexA from Escherichia coli (E. coli) (Brent and Ptashne (1985) Cell 43: 729-36 (PMID: 3907859)). In one preferred embodiment, the promoter comprises a minimal promoter operably linked to an upstream activation site comprising four DNA-binding domains of the yeast transcriptional activator GAL4. Schwechheimer et al. (1998) Plant Mol Biol 36: 195 -204 (PMID: 9484432).

Polyadenlation signals include, but are not limited to, the Agrobacterium octopine synthase signal (Gielen et al. (1984) EMBO J 3: 835-46 (PMID: 6327292)) and the nopaline synthase signal (Depicker et al. (1982) Mol and Appl Genet 1: 561-73 (PMID: 7153689)).

Transcriptional termination regions include, but are not limited to, the terminators of the A. tumefaciens Ti plasmid octopine synthase and nopaline synthase genes. See Ballas et al. (1989) Nuc Acid Res 17: 7891-903 (PMID: 2798133), Guerineau et al. (1991) Mol Gen Genet 262: 141-4 (PMID: 1709718), Joshi (1987) Nuc Acid Res 15: 9627-39 (PMID: 3697078), Mogen et al. (1990) Plant Cell 2: 1261-72 (PMID: 1983794), Munroe et al. (1990) Gene 91:151-8 (PMID: 1976572), Proudfoot (1991) Cell 64: 671-4 (PMID: 1671760), and Sanfacon et al. (1991) Genes Devel 5: 141-9 (PMID: 1703507). If translation of the transcript is desired, translational start and stop codons can also be provided.

The expression cassettes of the invention may be covalently linked to a polynucleotide encoding a selectable or screenable marker. Examples of such markers include genes encoding drug or herbicide resistance, such as hygromycin resistance (hygromycin phosphotransferase (HPT)), spectinomycin (encoded by the aada gene), kanamycin and gentamycin resistance (neomycin phosphotransferase (nptII)), streptomycin resistance (streptomycin phosphotransferase gene (SPT)), phosphinothricin or basta resistance (bamase (bar)), chlorsulfuron reistance (acetolactase synthase (ALS)), chloramphenicol resistance (chloramphenicol acetyl transferase (CAT)), G418 resistance, lincomycin resistance, methotrexate resistance, glyphosate resistance, and the like. In addition, the expression cassettes of the invention may be covalently linked to genes encoding enzymes that are easily assayed, for example, luciferase, alkaline phosphatase, β-galactosidase (β-gal), β-glucuronidase (GUS) and the like.

In one embodiment, the invention provides an expression cassette, comprising a polynucleotide encoding an antisense RNA that is complementary to a nucleic acid encoding either the polypeptide of SEQ ID NO: 2, 4, 6, 8 or 10, or a polypeptide having at least 80% sequence identity to SEQ ID NO: 2, 4, 6, 8 or 10, wherein said polynucleotide is operably linked to a promoter that can be active in a plant cell.

In a preferred embodiment, the antisense RNA comprises the complement of SEQ ID NO: 1, 7 and 9. In another preferred embodiment, the antisense RNA comprises the complement of SEQ ID NO: 3 or 5. In another embodiment, the antisense RNA has at least 80% sequence identity with at least 20 consecutive nucleotides of SEQ ID NO: 1, 3, 5, 7 or 9. In still another embodiment, the antisense RNA hybridizes under high stringency conditions to the polynucleotide of SEQ ID NO: 1, 3, 5, 7 or 9.

In another aspect, the invention provides vectors containing the expression cassettes of the invention. By “vector” is intended a polynucleotide sequence that is able to replicate in a host cell. Preferably the vector contains genes that serve as markers useful in the identification and/or selection of transformed cells. Such markers include, but are not limited to barnase (bar), G418, hygromycin, kanamycin, bleomycin, gentamicin and the like. The vector can comprise DNA or RNA and can be single or double stranded, and linear or circular. Various plant expression vectors and reporter genes are described in Gruber et al. in Methods in Plant Molecular Biology and Biotechnology, Glick et al., eds, CRC Press, pp.89-119, 1993; and Rogers et al. (1987) Meth Enzymol 153: 253-77. In a preferred embodiment, the vector is an E. coli/A. tumefaciens binary vector. Most preferably, the expression cassette is inserted between the right and left borders of a T-DNA from an Agrobacterium Ti plasmid.

Introduction of the polynucleotides of the invention (including expression cassettes and vectors) into a plant, plant cell or plant tissue will result in the modulation of plant growth. Thus, in one aspect, the invention provides plants, plant cells and plant tissues transformed with at least one polynucleotide, expression cassette or vector of the invention. By transformation is meant the introduction of a polynucleotide into a target plant cell or plant tissue.

Antisense polynucleotides, dsRNA and ribozymes can be introduced directly into plant cells, in the form of RNA. Alternatively, the antisense polynucleotides, dsRNA and ribozymes of the present invention may be provided as RNA via transcription in plant cells transformed with expression constructs encoding such RNAs.

In a preferred embodiment, a plant or plant cell is transformed with an expression cassette, comprising a polynucleotide encoding an antisense RNA that is complementary to a nucleic acid encoding either the polypeptide of SEQ ID NO: 2, 4, 6, 8 or 10, or a polypeptide having at least 80% sequence identity to SEQ ID NO: 2, 4, 6, 8 or 10, wherein said polynucleotide is operably linked to a promoter that can be active in a plant cell.

The polynucleotides of the invention may be introduced into any plant or plant cell. By plants is meant angiosperms (monocotyledons and dicotyledons) and gymnosperms, and the cells, organs and tissues thereof. Methods for the introduction of polynucleotides into plants and for generating transgenic plants are known to those skilled in the art. See, for example, Weissbach & Weissbach (1988) Methods for Plant Molecular Biology, Academic Press, N.Y. and Grierson & Corey (1988) Plant Molecular Biology, 2^{nd} Ed., Blackie, London, Miki et al. (1993) Procedures for Introducing foreign DNA into Plants, CRC Press, Inc. pp.67-80. Such methods include, but are not limited to electroporation (Fromm et al. (1985) Proc Natl Acad Sci 82: 5824 (PMID: 3862099) and Riggs et al. (1986) Proc Natl Acad Sci USA 83: 5602-6 (PMID: 3016708)), particle bombardment (U.S. Pat. Nos. 4,945,050 and 5,204,253, the contents of which are incorporated by reference, Klein et al. (1987) Nature 327: 70-3, McCabe et al. (1988) Biotechnology 6: 923-26), microinjection (Crossway (1985) Mol Gen Genet 202: 179-85 and Crossway et al. (1986) Biotechniques 4: 320-34), silicon carbide mediated DNA uptake (Kaeppler et al. (1990) Plant Cell Reporter 9: 415-18), direct gene transfer (Paszkowski et al. EMBO J 3: 2717-22), protoplast fusion (Fraley et al. (1982) Proc Natl Acad Sci USA 79: 1859-63), polyethylene glycol precipitation (Paszowski et al.(1984) EMBO J 3:2717-22 and Krens et al. (1982) Nature 296: 72-4), silicon fiber delivery, agroinfection (U.S. Pat. No. 5,188,958, incorporated herein by reference, Freeman et al. (1984) Plant Cell Physiol 25: 1353 (liposome mediated DNA uptake), Hinchee et al. (1988) Biotechnology 6: 915-21, Horsch et al. (1984) Science 233: 496-8, Fraley et al. (1983) Proc Natl Acad Sci USA 80: 4803, Hemalsteen et al. (1984) EMBO J 3: 3039-41, Hooykass-Van Sloteren et al. (1984) Nature 311: 763-4, Grimsley et al. (1987) Nature 325: 1677-9, Gould et al. (1991) Plant Physiol 95: 426-34, Kindle (1990) Proc Natl Acad Sci USA 87: 1228 (vortexing method), Bechtold et al. (1995) In Gene Transfer to Plants, Potrykus et al. (Eds) Springer-Verlag, New York, N.Y. pp19-23 (vacuum infiltration), Schell (1987) Science 237: 1176-83; and Plant Molecular Biology Manual, Gelvin and Schilperoort, eds., Kluwer, Dordrecht, 1994).

Preferably, the polynucleotides of the invention are introduced into a plant cell by agroinfection. In this method, a DNA construct comprising a polynucleotide of the invention is inserted between the right and left T-DNA borders in an Agrobacterium tumefaciens vector. The virulence proteins of the A. tumefaciens host cell will mediate the transfer of the inserted DNA into a plant cell infected with the bacterium. As an alternative to the A. tumefaciens/Ti plasmid system, Agrobacterium rhizogenes-mediated transformation may be used. See Lichtenstein and Fuller in: Genetic Engineering, Volume 6, Ribgy (ed) Academic Press, London, 1987; Lichtenstein and Draper, in DNA Cloning, Volume 2, Glover (ed) IRI Press, Oxford, 1985.

If one or more plant gametes are transformed, transgenic seeds and plants can be produced directly. For example, a preferred method of producing transgenic Arabidopsis seeds and plants involves agroinfection of the flowers and collection of the transgenic seeds produced from the agroinfected flowers. Alternatively, transformed plant cells can be regenerated into plants by methods known to those skilled in the art. See, for example, Evans et al, Handbook of Plant Cell Cultures, Vol I, MacMollan Publishing Co. New York, 1983; and Vasil, Cell Culture and Somatic Cell Genetics of Plants, Acad Press, Orlando, Vol II, 1986.

Once a transgenic plant has been obtained, it may be used as a parent to produce progeny plants and plant lines. Conventional plant breeding methods can be used, including, but not limited to crossing and backcrossing, self-pollination and vegetative propagation. Techniques for breeding plants are known to those skilled in the art. The progeny of a transgenic plant are included within the scope of the invention, provided that the progeny contain all or part of the transgenic construct.

The transformed plants and plant cells of the invention include the progeny of said plant or plant cell, as long as the progeny plants or plant cells still contain the antisense expression cassette. Progeny may be generated by both asexual and sexual methods. Progeny of a plant include seeds, subsequent generations of the plant and the seeds thereof.

Introduction of the polynucleotides of the invention into a plant, plant cell or plant tissue will result in the modulation of plant growth or development. In most cases, the modulation will be a decrease or cessation of growth or development of the plant cells or tissues where the polynucleotides of the invention are expressed.

The antisense, ribozymes, dsRNA and sense polynucleotides of the invention may be directly transformed into a plant cell. Alternatively, the expression cassettes or vectors of the invention may be introduced into a plant cell. Once in the cell, expression of the antisense, ribozymes, dsRNA and sense polynucleotides of the invention may be transient or stable. Stable expression requires that all or a part of the polynucleotide, expression cassette or vector is integrated into a plant chromosome or a stable extra-chromosomal replicon.

Thus, in one embodiment, the invention provides a method for, modulating plant growth and/or development, comprising:

a) introducing into a plant or plant cell at least one RNA polynucleotide, wherein said RNA polynucleotide is selected from the group consisting of:

i) an RNA complementary to SEQ ID NO: 1, 3, 5, 7 or 9;

ii) an RNA complementary to at least 20 consecutive nucleotides of SEQ ID NO: 1, 3, 5, 7 or 9;

iii) an RNA complementary to a nucleic acid having at least 80% sequence identity with SEQ ID NO: 1, 3, 5, 7 or 9;

iv) an RNA complementary to at least 30 consecutive nucleotides of a nucleic acid encoding SEQ ID NO: 2, 4, 6, 8 or 10;

v) an RNA complementary to a nucleic acid encoding a polypeptide having at least 80% sequence identity with SEQ ID NO: 2, 4, 6, 8 or 10;

vi) a ribozyme specific for a nucleic acid encoding a polypeptide having at least 80% sequence identity with SEQ ID NO: 2, 4, 6, 8 or 10;

vii) a dsRNA specific for a nucleic acid encoding a polypeptide having at least 80% sequence identity with SEQ ID NO: 2, 4, 6, 8 or 10;

viii) an RNA having at least 80% sequence identity with SEQ ID NO: 1, 3, 5, 7 or 9; and

iv) an RNA encoding a polypeptide having at least 80% sequence identity with SEQ ID NO: 2, 4, 6, 8 or 10; and

b) selecting said plant or plant cell expressing said RNA polynucleotide;

wherein said plant growth and/or development is decreased or altered.

In another embodiment, the invention provides a method for modulating the growth and/or development of a plant, plant cell or plant tissue, comprising: transforming said plant, plant cell or plant tissue with an expression cassette comprising a polynucleotide encoding a sense RNA encoding either the polypeptide of SEQ ID NO: 2, 4, 6, 8 or 10, or a polypeptide having at least 80% sequence identity to SEQ ID NO: 2, 4, 6, 8 or 10, wherein said polynucleotide encoding said sense RNA is operably linked to a promoter that can be active in a plant cell. In a preferred embodiment, the promoter is a tissue specific promoter.

In another embodiment, the invention provides a method for modulating the growth and/or development of a plant, plant cell or plant tissue, comprising: transforming said plant, plant cell or plant tissue with at least one expression cassette, wherein said expression cassette(s) comprise(s) the polynucleotides encoding a dsRNA that is specific for a nucleic acid encoding either the polypeptide of SEQ ID NO: 2, 4, 6, 8 or 10, or a polypeptide having at least 80% sequence identity to SEQ ID NO: 2, 4, 6, 8 or 10, wherein said polynucleotides are operably linked to a promoter that can be active in a plant cell. In a preferred embodiment, the promoter is a tissue specific promoter.

In yet another embodiment, the invention provides a method for modulating the growth and/or development of a plant, plant cell or plant tissue, comprising: transforming said plant, plant cell or plant tissue with an expression cassette comprising a polynucleotide encoding a ribozyme specific for a nucleic acid encoding either the polypeptide of SEQ ID NO: 2, 4, 6, 8 or 10, or a polypeptide having at least 80% sequence identity to SEQ ID NO: 2, 4, 6, 8 or 10, wherein said polynucleotide is operably linked to a promoter that can be active in a plant cell. In a preferred embodiment, the promoter is a tissue specific promoter.

In a preferred embodiment, the invention provides a method for modulating the growth and/or development of a plant, plant cell or plant tissue, comprising: transforming said plant, plant cell or plant tissue with an expression cassette comprising a polynucleotide encoding an antisense RNA that is complementary to a nucleic acid encoding either the polypeptide of SEQ ID NO: 2, 4, 6, 8 or 10, or a polypeptide having at least 80% sequence identity to SEQ ID NO: 2, 4, 6, 8 or 10, wherein said polypeptide is operably linked to a promoter that can be active in a plant cell. In a preferred embodiment, the promoter is a tissue specific promoter.

Male tissue-preferred expression of any of these RNAs in one or more male tissues can result in a male sterile plant. In general, the plant progeny obtained by cross-pollination show more vigor than the progeny obtained through self-pollination.

Thus, the invention provides a method for generating a male sterile plant, comprising:

a) transforming a plant cell with an expression cassette selected from the group consisting of:

i) an expression cassette comprising a polynucleotide encoding an antisense RNA complementary to either a nucleic acid encoding the polypeptide of SEQ ID NO: 2, 4, 6, 8 or 10 or a nucleic acid encoding a polypeptide having at least 80% sequence identity with SEQ ID NO: 2, 4, 6, 8 or 10; wherein said polynucleotide is operably linked to a plant male tissue-preferred promoter;

ii) an expression cassette comprising a polynucleotide encoding a sense RNA encoding either the polypeptide of SEQ ID NO: 2, 4, 6, 8 or 10 or a polypeptide having at least 80% sequence identity with SEQ ID NO: 2, 4, 6, 8 or 10; wherein said polynucleotide is operably linked to a plant male tissue-preferred promoter; and

iii) at least one expression cassette, wherein said expression cassette(s) comprise(s) the polynucleotides encoding a dsRNA that is specific for a nucleic acid encoding the polypeptide of SEQ ID NO: 2,4, 6, 8 or 10 or a polypeptide having at least 80% sequence identity with SEQ ID NO: 2, 4, 6, 8 or 10; wherein said polynucleotides are operably linked to a plant male tissue-preferred promoter; and

b) obtaining a male sterile plant from said transformed plant cell.

In one embodiment, the male-tissue preferred promoter is a pollen-preferred promoter.

Ovule-preferred expression of any of the RNAs of the invention will result in a reduction of seed size. By “reduced seed size” is meant that the seed is reduced by at least 10%. Preferably, the seed is reduced in size to 25%, 50%, 75%, 90% or is absent. The seed of any plant may be reduced in size, however preferred plants include cucumbers, tomatoes, melons, cherries, grapes, pomegranates and the like.

Thus, the invention provides a method for generating a plant with reduced seed size, comprising:

a) transforming a plant cell with an expression cassette selected from the group consisting of:

i) an expression cassette comprising a polynucleotide encoding an antisense RNA complementary to either a nucleic acid encoding the polypeptide of SEQ ID NO: 2, 4, 6, 8 or 10 or a nucleic acid encoding a polypeptide having at least 80% sequence identity with SEQ ID NO: 2, 4, 6, 8 or 10; wherein said polynucleotide is operably linked to an ovule-preferred promoter;

ii) an expression cassette comprising a polynucleotide encoding a sense RNA encoding either the polypeptide of SEQ ID NO: 2, 4, 6, 8 or 10 or a polypeptide having at least 80% sequence identity with SEQ ID NO: 2, 4, 6, 8 or 10; wherein said polynucleotide is operably linked to an ovule-preferred promoter; and

iii) at least one expression cassette, wherein said expression cassette(s) comprise(s) the polynucleotides encoding a dsRNA that is specific for a nucleic acid encoding the polypeptide of SEQ ID NO: 2, 4, 6, 8 or 10 or a polypeptide having at least 80% sequence identity with SEQ ID NO: 2, 4, 6, 8 or 10; wherein said polynucleotides are operably linked to an ovule-preferred promoter; and

b) obtaining a plant having reduced seed size from said transformed plant cell.

In another aspect the invention provides methods for identifying compounds as herbicide candidates by determining the difference in activity of a germin-like protein in the presense and absence of the compound. One method comprises measuring the auxin-binding activity of a germin-like protein in the absence and presense of the compound. Determining the difference in the auxin-binding activity in the presense and absense of the compound, wherein a difference in the auxin-binding activity indicates the compound as a herbicide candidate. In one embodiment the method comprises contacting a germin-like protein with fluorescein-indole acetic acid in the absence and presense of the compound. Measuring the fluorescence polarization in the presense and absense of the compound, wherein a difference in the fluorescence polarization in the presense and absense of the compound indicates the compound as a herbicide candidate. In one embodiment the germin-like protein is SEQ ID NO: 2, 4, 6, 8 or 10.

In another aspect of the invention, a method for identifying a compound as a candidate for a herbicide comprises measuring the oxalate oxidase activity of a germin-like protein in the absence and presense of the compound. Determining the difference in the oxalate oxidase activity in the presense and absense of the compound, wherein a difference in the oxalate oxidase activity indicates the compound as a herbicide candidate. In one embodiment the method comprises contacting a germin-like protein with oxalate in the absence and presense of the compound. Measuring the H₂O₂ produced in the presense and absense of the compound, wherein a difference in the amount of H₂O₂ in the presense and absense of the compound indicates the compound as a herbicide candidate. In one embodiment the germin-like protein is SEQ ID NO: 2, 4, 6, 8 or 10.

In another aspect of the invention, a method for identifying a compound as a candidate for a herbicide comprises measuring the ADPG pyrophosphatase activity of a germin-like protein in the absence and presense of the compound. Determining the difference in ADPG pyrophosphatase activity in the presense and absense of the compound, wherein a difference in the ADPG pyrophosphatase activity indicates the compound as a herbicide candidate. In one embodiment the germin-like protein is SEQ ID NO: 2, 4, 6, 8 or 10.

### EXAMPLE 1

### Construction of Transgenic Plants Expressing the Driver

The “Driver” is an artificial transcription factor comprising a chimera of the DNA-binding domain of the yeast GAL4 protein (amino acid residues 1-137) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained according to the procedures described below.

Plant Growth Conditions

Unless, otherwise indicated, all plants were grown Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ in down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻²s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 2

### Construction of Antisense Expression Cassettes in a Binary Vector

A fragment of an Arabidopsis thaliana cDNA corresponding to SEQ ID NO: 1, 7 and 9, 7 and 9 was each ligated into the PacI/AscI sites of an E. coli/Agrobacterium binary vector in the antisense orientation. This placed transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E. coli. Kanamycin resistant clones were selected and purified. DNA was isolated from each clone and characterized by PCR and sequence analysis. The antisense expression cassette and a constitutive chemical resistance expression cassette are located between right and left T-DNA borders. Thus, the antisense expression cassettes can be transferred into a recipient plant cell by agroinfection.

### EXAMPLE 3

### Transformation of Agrobacterium with the Antisense Expression Cassette

The antisense expression cassettes of Example 2 were transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of an Arabidopsis Antisense Target Plants

The antisense expression cassettes were introduced into Arabidopsis thaliana wild-type plants by the following method. Five days prior to agroinfection, the primary inflorescence of Arabidopsis thaliana plants grown in 2.5 inch pots were clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the antisense expression cassettes. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome in order to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial.

One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represent stably transformed T1 plants.

### EXAMPLE 5

### Effect of pPG329, pPg50710, and pPg50704 Antisense Expression in Arabidopsis Seedlings

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The transgenic plant lines containing the pPG329, pPg50710, and pPg50704 antisense constructs exhibited an absence of leaf growth and delayed development, chlorosis and reduced size, and short roots and reduced size, respectively.

The data from the antisense lines expressing pPG329, pPg50701, and pPg50704 demonstrates that the antisense expression of these sequences results in significantly impaired growth. Thus, sense sequence corresponding to pPG329, pPg50701, and pPg50704 and protein encoded by these sequences is essential for normal plant growth and development.

### EXAMPLE 6

### Cloning & Expression Strategies, Extraction and Purification of the Germin-Like Proteins

The following protocol may be employed to obtain purified germin-like protein.

Cloning and expression strategies:

A gene encoding a germin-like protein is cloned into E. coli (pET vectors-Novagen), Baculovirus (Pharmingen) and Yeast (Invitrogen) expression vectors containing His/fusion protein tags. SDS-PAGE and Western blot analysis is used to evaluate recombinant protein expression.

Extraction:

Extract recombinant protein from 250 ml cell pellet in 3 mL of extraction buffer by sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni—NTA affinity chromatography (Qiagen).

Purification protocol: perform all steps at 4° C.:

Use 3 ml Ni-beads (Qiagen)

Equilibrate column with the buffer

Load protein extract

Wash with the equilibration buffer

Elute bound protein with 0.5 M imidazole

### EXAMPLE 7

### Assays for Testing Inhibitors or Candidates for Inhibition of Germin-Like Protein Activity

The enzymatic activity of the germin-like proteins of the invention may be determined in the presence and absence of candidate inhibitors in a suitable reaction mixture, such as described by any of the following known assay protocols:

A. Oxalate oxidase assay:

Oxalate oxidase activity can be monitored using the Western blotting technique described by Zhang et al. (1996) Plant Molecular Biology Reporter 14:266-72. Briefly, water extracts of frozen tissue powders can be loaded onto an SDS-PAGE gel in a loading buffer lacking reducing agent and without boiling. Proteins can then be blotted onto nitrocellulose, with subsequent immunodetection of proteins on the blot performed according to standard procedures.

B. In Situ Detection of Oxalate Oxidase activity:

Leaf specimens, harvested 24 hours after inoculation with C¹⁵, can be incubated at room temperature in an oxalate oxidase activity developer solution (40 mm succinic acid/NaOH, pH 3.5, 2 mm oxalic acid, 0.5 mg/mL 4-chloro-4-naphthol, and 3.5 mm EDTA) for in situ activity detection. The stained speciments can then be fixed in 4% paraformaldehyde in PBS (130 mm NaCl, 7 mm Na₂HPO₄, and 3 mm NaH₂PO₄, pH 7.0). After being washed in PBS, the specimens can be infiltrated in a series of gelatin solutions (5-20%) in PBS at 40 degrees Celsius and embedded in 20% gelatin. The blocks are frozen to −20 degrees Celsius and stabilized with ice, and 30-μm sections can be made by cryostat-sectioning in a rotary retracting microtome (such as model 5030, Bright, Huntingdon, UK). Sections can then be examined by light microsopy and photographed. Samples positive for oxalate oxidase activity will appear with a dark-blue stain. Please see Zhou et al. (1998) Plant Physiology 117:33-41 and Dumas et al. (1995) Plant Physiology 107:1091 -1096.

C. Oxalate oxidase assay:

Oxalate oxidase activity is assayed by monitoring the production of H₂O₂ upon incubation of the germin-like protein with oxalate. A number of methods for measuring H₂O₂ are known in the art. In one example, measurement of H₂O₂ is performed by monitoring the absorbance at 590 nm produced upon reaction of H₂O₂ with 3-methyl-2-benzothiozolinone hydrazone (MBTH) and 3-(dimethylamino) benzoic acid (DMAB) in the presence of peroxidase (Sigma Kit No. 591 -C). In another example, measurement of H₂O₂ is performed by monitoring the absorbance at 520 nm produced upon reaction of H₂O₂ with 4-aminophenazone and phenol in the presence of peroxidase (Sigma No. P6782).

D. Auxin binding assay:

To assay auxin-binding, fluorescein-indole acetic acid is first produced by reacting fluorescein cadaverine (Molecular Probes No. A-1 0466) with 1 equivalent of indole-3-acetic acetic acid (Acros No. 122160100) in anhydrous dimethyl sulfoxide in the presence of 1-ethyl-3-(3dimethylaminopropyl)carbodiimide (EDC) (Sigma No. H7377) and N-hydroxysuccinimide (NHS) (Sigma No. H7377). The product fluorescein-indole acetic acid is purified by flash chromatography on silica column. Fluorescein-indole acetic acid s incubated with the recombinant germin-like protein. Fluorescence polarization is measured on Ultra using the fluorescein polarization filter set: 335 nm excitation, 485 nm emission.

E. ADPG pyrophosphatase assay:

ADPG pyrophosphatase activity is measured by linking with phosphoglucomutase (Sigma No. P6156) and glucose-6-phosphate dehydrogenase (Sigma No. G8404). ADPG (adenosine-5′-diphosphoglucose) is available from Sigma, Catalogue No. A0627. NADH production can be monitored by any of a variety of means known in the art.

While the foregoing describes certain embodiments of the invention, it will be understood by those skilled in the art that variations and modifications may be made and still fall within the scope of the invention.

1. A method for identifying a compound as a candidate for a herbicide, comprising:
a) contacting said compound with a polypeptide selected from the group consisting of:
i) the polypeptide of SEQ ID NO: 2, 4, 6, 8 or 10; and
ii) a polypeptide having at least 80% sequence identity with the polypeptide of SEQ ID NO: 2, 8 or 10; and
b) detecting the presence and/or absence of binding between said compound and said polypeptide; wherein binding indicates that said compound is a candidate for a herbicide.

2. The method of claim 1, wherein said polypeptide is the polypeptide of SEQ ID NO: 2.

3. The method of claim 1, wherein said polypeptide has at least 90% sequence identity with the polypeptide of SEQ ID NO: 2.

4. The method of claim 1, wherein said polypeptide has at least 95% sequence identity with the polypeptide of SEQ ID NO: 2.

5. A method for generating a male sterile plant, comprising:
a) transforming a plant cell with the expression cassette of claim 29; and
b) obtaining said male sterile plant from said transformed plant cell.

6. A method for generating a plant that produces seedless fruits, comprising:
a) transforming a plant cell with the expression cassette of claim 31; and
b) obtaining said plant that produces seedless fruits from said transformed plant cell.

7. A method for modulating plant growth and/or development comprising:
a) introducing into a plant or plant cell at least one RNA polynucleotide, said RNA polynucleotide selected from the group consisting of:
i) an RNA complementary to SEQ ID NO: 1, 3, 5, 7 or 9;
ii) an RNA complementary to at least 20 consecutive nucleotides of SEQ ID NO: 1, 3, 5, 7 or 9;
iii) an RNA complementary to a nucleic acid having at least 80% sequence identity with SEQ ID NO: 1, 7 or 9;
iv) an RNA complementary to at least 30 consecutive nucleotides of a nucleic acid encoding SEQ ID NO: 2, 4, 6, 8 or 10;
v) an RNA complementary to a nucleic acid encoding a polypeptide having at least 80% sequence identity with SEQ ID NO: 2;
vi) a ribozyme specific for a nucleic acid encoding a polypeptide having at least 80% sequence identity with SEQ ID NO: 2;
vii) a dsRNA specific for a nucleic acid encoding a polypeptide having at least 80% sequence identity with SEQ ID NO: 2;
viii) an RNA having at least 80% sequence identity with SEQ ID NO: 1, 7 or 9; and
ix) an RNA encoding a polypeptide having at least 80% sequence identity with SEQ ID NO: 2; and
b) selecting said plant or plant cell expressing said RNA polynucleotide; wherein said plant growth and/or development is decreased or altered.

8. A method for identifying a compound as a candidate for a herbicide, comprising:
a) contacting a germin-like protein with fluorescein-indole acetic acid in the absence of the compound;
b) contacting the germin-like protein with fluorescein-indole acetic acid in the presence of said compound; and
c) measuring the fluorescence polarization after the contacting of steps (a) and (b), wherein a difference in the fluorescence polarization between steps (a) and (b) indicates the compound as a herbicide candidate.

9. A method for identifying a compound as a candidate for a herbicide, comprising:
a) measuring the auxin binding activity of a germin-like protein in the absence of the compound;
b) measuring the auxin binding activity of a germin-like protein in the presence of the compound; and
c) determining the difference in auxin binding activity between steps (a) and (b), wherein a difference in activity indicates the compound as a herbicide candidate.

10. A method for identifying a compound as a candidate for a herbicide, comprising:
a) contacting a protein selected from the group consisting of: SEQ ID NO: 2, 8 and 10 with fluorescein-indole acetic acid in the absence of the compound;
b) contacting the protein with fluorescein-indole acetic acid in the presence of said compound; and
c) measuring the fluorescence polarization after the contacting of steps (a) and (b), wherein a difference in the fluorescence polarization between steps (a) and (b) indicates the compound as a herbicide candidate.

11. A method for identifying a compound as a candidate for a herbicide, comprising:
a) measuring the auxin binding activity of a protein selected from the group consisting of: SEQ ID NO: 2, 8 and 10 in the absence of the compound;
b) measuring the auxin binding activity of the protein in the presence of the compound; and
c) determining the difference in auxin binding activity between steps (a) and (b), wherein a difference in activity indicates the compound as a herbicide candidate.

12. A method for identifying a compound as a candidate for a herbicide, comprising:
a) contacting a germin-like protein with oxalate in the absence of the compound;
b) contacting the germin-like protein with oxalate in the presence of the compound; and
c) measuring the H₂O₂ produced after the contacting of steps (a) and (b), wherein a difference in the amount of H₂O₂ between steps (a) and (b) indicates the compound as a herbicide candidate.

13. A method for identifying a compound as a candidate for a herbicide, comprising:
a) measuring the oxalate oxidase activity of a germin-like protein in the absence of the compound;
b) measuring the oxalate oxidase activity of the germin-like protein in the presence of the compound; and
c) determining the difference in oxalate oxidase activity between steps (a) and (b), wherein a difference in activity indicates the compound as a herbicide candidate.

14. A method for identifying a compound as a candidate for a herbicide, comprising:
a) contacting a protein selected from the group consisting of: SEQ ID NO: 2, 8 and 10 with oxalate in the absence of the compound;
b) contacting the protein with oxalate in the presence of said compound; and
c) measuring the H₂O₂ produced after the contacting of steps (a) and (b), wherein a difference in the amount of H₂O₂ between steps (a) and (b) indicates the compound as a herbicide candidate.

15. A method for identifying a compound as a candidate for a herbicide, comprising:
a) measuring the oxalate oxidase activity of a protein selected from the group consisting of: SEQ ID NO: 2, 8 and 10 in the absence of the compound;
b) measuring the oxalate oxidase activity of the protein in the presence of the compound; and
c) determining the difference in oxalate oxidase activity between steps (a) and (b), wherein a difference in activity indicates the compound as a herbicide candidate.

16. A method for identifying a compound as a candidate for a herbicide, comprising:
a) measuring the ADPG pyrophosphatase activity of a germin-like protein in the absence of the compound;
b) measuring the ADPG pyrophosphatase activity of the germin-like protein in the presence of the compound; and
c) determining the difference in ADPG pyrophosphatase activity between steps (a) and (b), wherein a difference in activity indicates the compound as a herbicide candidate.

17. A method for identifying a compound as a candidate for a herbicide, comprising:
a) measuring the ADPG pyrophosphatase activity of a protein selected from the group consisting of: SEQ ID NO: 2, 8 and 10 in the absence of the compound;
b) measuring the ADPG pyrophosphatase activity of the protein in the presence of the compound; and
c) determining the difference in ADPG pyrophosphatase activity between steps (a) and (b), wherein a difference in activity indicates the compound as a herbicide candidate.


SEQUENCE LISTING (10 sequences)

SEQ ID NO: 1 - DNA, 627 bp, Arabidopsis thaliana
atgttgcgtactatcttcctcttatctcttctctttgctctatccaatgcctctgttcaagatttctgtgtcgcaaacctgaaacgcgctgaaacccctgcgggttacccttgcattcgtcccattcatgtcaaagctacagactttgtcttctctggcttaggcactcctggaaacactacaaacatcatcaacgccgctgtcacacccgctttcgcagctcagttcccgggtctgaacggtctaggcctctctacagctagacttgacttagctcctaaaggtgtgatcccaatgcacactcaccctggtgcctctgaggttctctttgtccttactggctccattaccgctgggtttgtctcctcggcaaacgctgtctacgtgcagacactcaaaccaggacaggtcatggttttcccacagggcttgcttcatttccagatcaacgcgggaaaatcctctgcttcagccgttgtcactttcaacagcgctaatccgggtctgcagattctcgacttcgcactctttgctaacagtcttcccactgaactcgtcgtgggtactactttccttgacgccactacagtcaagaagctaaagggtgttcttggaggaactggctaa

SEQ ID NO: 2 - protein, 208 aa, Arabidopsis thaliana
MLRTIFLLSLLFALSNASVQDFCVANLKRAETPAGYPCIRPIHVKATDFVFSGLGTPGNTTNIINAAVTPAFAAQFPGLNGLGLSTARLDLAPKGVIPMHTHPGASEVLFVLTGSITAGFVSSANAVYVQTLKPGQVMVFPQGLLHFQINAGKSSASAVVTFNSANPGLQILDFALFANSLPTELVVGTTFLDATTVKKLKGVLGGTG

SEQ ID NO: 3 - DNA, 627 bp, Arabidopsis thaliana
atgttgcgtactatcttcctcttatctcttctctttggtctatccaatgcctctgttcaagatttctgtgtcgcaaacctgaaacgcgctgaaacccctgcgggttacccttgcattcgtcccattcatgtcaaagctacagactttgtcttctctggcttaggcactcctggaaacactacaaacatcatcaacgccgctgtcacacccgctttcgcagctcagttcccgggtctgaacggtctagccctctctacagctagacttgacttagctcctaaaggtgtgatcccaatgcacactcaccctggtgcctctgaggttctctttgtccttactggctccattaccgctgggtttgtctcctcggcaaacgctgtctacgtgcagacactcaaaccaggacaggtcatggttttcccacagggcttgcttcatttccagatcaacgcgggaaaatcctctgcttcagccgttgtcactttcaacagcgctaatcggggtctgcagattctcgacttcgcactctttgctaacagtcttcccactgaactcgtcgtgggtactactttccttgacgccactacagtcaagaagctaaagggtgttcttggaggaactggctaa

SEQ ID NO: 4 - protein, 208 aa, Arabidopsis thaliana
MLRTIFLLSLLFGLSNASVQDFCVANLKRAETPAGYPCIRPIHVKATDFVFSGLGTPGNTTNIINAAVTPAFAAQFPGLNGLALSTARLDLAPKGVIPMHTHPGASEVLFVLTGSITAGFVSSANAVYVQTLKPGQVMVFPQGLLHFQINAGKSSASAVVTFNSANRGLQILDFALFANSLPTELVVGTTFLDATTVKKLKGVLGGTG

SEQ ID NO: 5 - DNA, 624 bp, Brassica napus
atgttgcgcattatcttcctcttgtctctcctcttcgctctctccaatgactcagttcaagacttctgcgtcgccaacctcaaacgcgctgagacccccgctggctacccttgcatccgccccatccacgtcaaagcctcggacttcgtcttcagcttaggcactcctggtaacaccaccaacatcatcagcgccgcggtgacaccaggcttcgtcgctcagttcccggctctgaacggtctaggcatctctactgctaggcttgacctagcacctaaaggtgtgatcccaatgcacactcaccctggcgcctctgaggttctcttcgtcctcgacggctctatcaccgctggattcatctcctctgccaactctgtctacgtgcagacgcttaaaccgggacaggtcatggtgttcccgcagggcttgcttcatttccagatcaatgctggtaaaacccctgctgctgcgttggtcactttcagcagtgcgagtcctggtctccagattcttgactttgcgctatttgctaatactctttccactgaactcgtctcagctactactttcctaccgcctgctacagtcaagacgcttaagggtgttcttggtggaactggctaa

SEQ ID NO: 6 - protein, 207 aa, Brassica napus
MLRIIFLLSLLFALSNDSVQDFCVANLKRAETPAGYPCIRPIHVKASDFVFSLGTPGNTTNIISAAVTPGFVAQFPALNGLGISTARLDLAPKGVIPMHTHPGASEVLFVLDGSITAGFISSANSVYVQTLKPGQVMVFPQGLLHFQINAGKTPAAALVTFSSASPGLQILDFALFANTLSTELVSATTFLPPATVKTLKGVLGGTG

SEQ ID NO: 7 - DNA, 669 bp, Arabidopsis thaliana
atgaggttttccaagtctctcatcctgattaccttatcggctttggtcatttcctttgccgaagctaatgatccaagtccacttcaagacttttgtgtggccattggcgacctcaaaaatggtgtttttgtgaatggtaagttttgcaaggatccaaagcaagcaaaggcagaagatttcttttactcaggcctcaaccaagcaggaaccactaataataaagtcaaatccaacgtgacaacagtcaatgtcgatcagattccagggttaaacactttgggaatatccttggtccgcatagactatgcgccatatggtcaaaacccgcctcacacacaccctcgtgccactgagatccttgttcttgttgagggaacattatatgttggttttgtctcttccaatcaagacaacaaccgtttattcgctaaagtgctgaacccgggcgacgtgtttgtgttccccataggaatgatccattttcaagtgaatatcgggaagacccctgcagtggcctttgctggactaagtagtcaaaatgctggtgtcatcacgattgcagatactgtgtttgggtcaacgcctccgattaatccagatattttggctcaggcgtttcagttagacgtcaatgttgttaaagaccttgaggccaagtttaaaaactaa

SEQ ID NO: 8 - protein, 222 aa, Arabidopsis thaliana
MRFSKSLILITLSALVISFAEANDPSPLQDFCVAIGDLKNGVFVNGKFCKDPKQAKAEDFFYSGLNQAGTTNNKVKSNVTTVNVDQIPGLNTLGISLVRIDYAPYGQNPPHTHPRATEILVLVEGTLYVGFVSSNQDNNRLFAKVLNPGDVFVFPIGMIHFQVNIGKTPAVAFAGLSSQNAGVITIADTVFGSTPPINPDILAQAFQLDVNVVKDLEAKFKN

SEQ ID NO: 9 - DNA, 633 bp, Arabidopsis thaliana
atgaagttcttcgtcgtgatcgtgttttgtgcaatcttcttatctgtctctggggattcggacaatatgcaggacacatgtcccacggctccgggagaacagagcatcttcttcatcaacggctatccttgcaagaacccgaccaagattaccgctcaggatttcaagtccaccaaacttacagaagctggagatacagacaattatctccagtcgaatgtcacattgctcactgcattagagtttccaggtctcaacactcttggcctctcggtctcacggactgatcttgaaagggacggatctgtgccgttccattcgcatccgaggtcatctgagatgctctttgtggtcaaaggagtcgtgtttgctggatttgtggatactaacaacaagatttttcaaacggttctgcaaaaaggcgatgtttttgtcttccctaaaggattgcttcatttctgcttgagcggtggctttgaaccagccaccgctttctcgttttacaatagccagaatcctggagtcgtgaatattggagaagtttttgggatcgatcaagagcatataaagatcatgacgaggtgtttagctactggctctggctgtagggtcactgacggtgatgagctttag

SEQ ID NO: 10 - protein, 210 aa, Arabidopsis thaliana
MKFFVVIVFCAIFLSVSGDSDNMQDTCPTAPGEQSIFFINGYPCKNPTKITAQDFKSTKLTEAGDTDNYLQSNVTLLTALEFPGLNTLGLSVSRTDLERDGSVPFHSHPRSSEMLFVVKGVVFAGFVDTNNKIFQTVLQKGDVFVFPKGLLHFCLSGGFEPATAFSFYNSQNPGVVNIGEVFGIDQEHIKIMTRCLATGSGCRVTDGDEL